(19) United States
(12) Plant Patent Application Publication
(10) Pub. No.: US 2004/0083512 P1
(43) Pub. Date: Apr. 29, 2004

(54) GRAPEVINE PLANT NAMED '13-21-12'

(76) Inventor: Timothy F. Bourne, Visalia, CA (US)

Correspondence Address: JONDLE & ASSOCIATES P.C., 9085 EAST MINERAL CIRCLE, SUITE 200, CENTENNIAL, CO, 80112, US

(21) Appl. No.: 10/280,675

(22) Filed: Oct. 24, 2002

(51) Int. Cl. A01H 5/00

(52) U.S. Cl. PLT/207

(57) ABSTRACT

This new variety ‘13-21-12’ is new and different because of its unusual shape, late ripening and very large berry size. It differs from the ‘Thompson Seedless’ variety, from which it descends, by having a much larger berry size, berries with a tougher skin and looser cluster. It somewhat resembles the ‘Calmeria’ variety from which it also descends by having a seedless berry and larger berry size.

### GENUS AND SPECIES

Vitis vinifera

### BACKGROUND AND SUMMARY OF THE INVENTION

The new table grapevine ‘13-21-12’ is of Vitis vinifera parentage and resulted from a twenty year breeding program which had as its goal the development of a late ripening, white, seedless table grape. The female, seeded parent was the variety ‘Calmeria’ (unpatented). The male parent of the cross is an unnamed, seedless grapevine resulting from a series of crosses involving ‘Emperor’, ‘Thompson Seedless’, and ‘Calmeria’. The hybridization resulting in ‘13-21-12’ was made near McFarland, Calif. in 1992. The seedling was selected from a population of 218 seedlings of like parentage in 1996. It was then propagated by cuttings and grafting to ‘Freedom’ rootstock. Those resulting plants were stable and typical of the original vine.

The new grapevine variety, cv. ‘13-21-12’ somewhat resembles the standard variety ‘Thompson Seedless’, but differs from it by having much greater fruitfulness. While ‘Thompson Seedless’ requires cane pruning to achieve a crop, the new variety ‘13-21-12’ is very fruitful from its basal buds, allowing a full crop from spur pruned vines, each shoot yielding one or two large clusters of fruit. The new variety has a much tougher berry skin than ‘Thompson Seedless’, making it much more resistant to handling damage after harvest. Additionally, the ripening date of ‘13-21-12’ is generally about two weeks later than ‘Thompson Seedless’.

### DESCRIPTION OF THE FIGURES

The accompanying drawings illustrate the following:

FIG. 1: Fruit cluster at harvest.

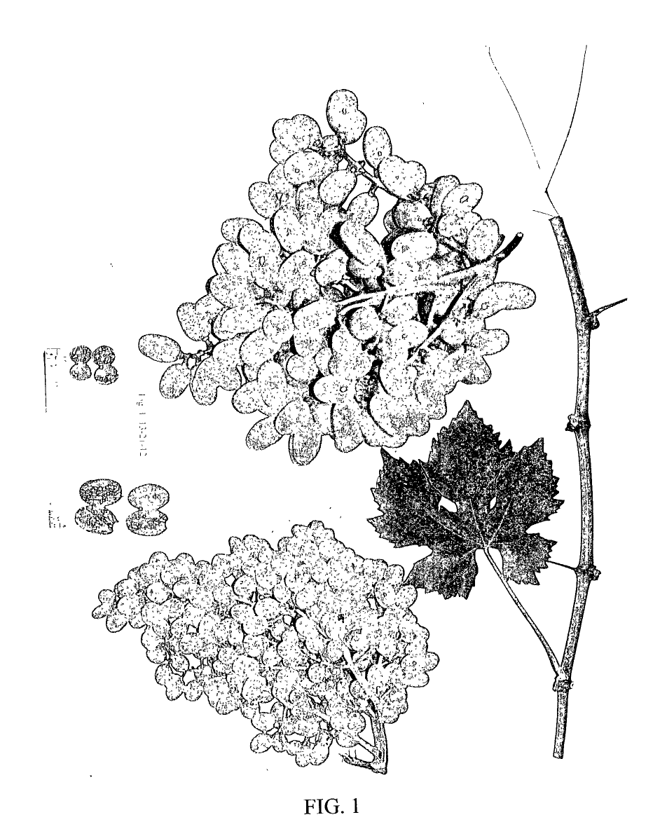

FIG. 2: Cane, leaf, natural fruit cluster (left) and fruit cluster following gibberellic acid applications (right).

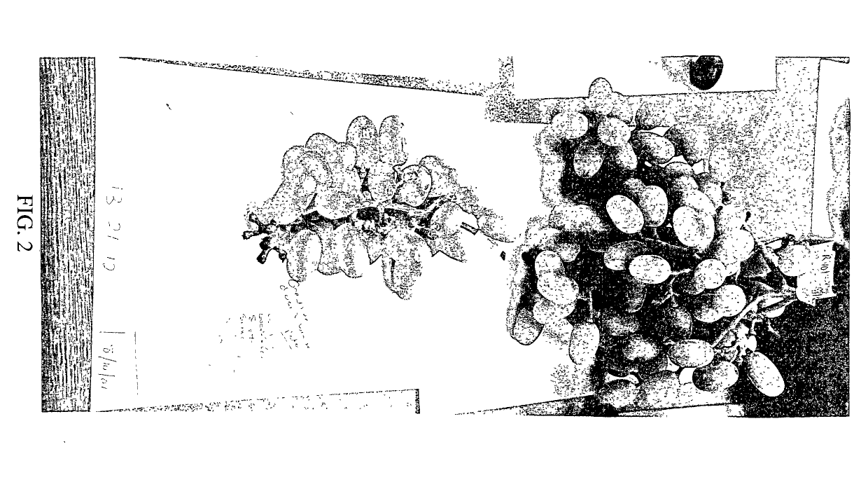

### DETAILED BOTANICAL DESCRIPTION OF THE INVENTION

The following description of grapevine ‘13-21-12’ contains references to color names taken from the Munsell Color Chart for Plant Tissues, published by Munsell Color, New Windsor, N.Y. Descriptors used herein conform to those set forth by the International Board for Plant Genetic Resources Institute Grape Descriptors (Vitis spp.) of 1983 and/or 1997 which were developed in collaboration with the Office International de la Vigne et du Vin (OIV) and the International Union for the Protection of New Varieties of Plants (UPOV).

Descriptions of the new invention apply to vines of ‘13-21-12’ grown on ‘Freedom’ rootstock at a density of 1,537 vines per hectare grown in Kern County, California in 2001. These vines were)in their first year of full production having been planted in 1998. These descriptions are believed to apply generally to the new variety grown under similar circumstances elsewhere:

### VINE

General:

Size.—Large.

Vigor.—High.

Density of foliage.—Dense.

Productivity.—Very productive when spur pruned, up to 58,627 kg./hectare.

Trunk circumference.—20.2 cm. at 1 meter height.

Rootstock.—Freedom.

Trunk:

Shape.—Medium.

Straps.—Long, split.

Surface texture.—Shaggy.

Inner bark color.—Brown 5YR 4/8.

### LEAVES

Mature leaves:

Average blade length.—14.6 cm.

Average blade width.—18.2 cm.

Size of blade.—Medium to large.

Shape.—Pentagonal.

Anthocyanin coloration of main veins on the upper side of the blade.—Absent.

Mature leaf profile.—Flat.

Blistering surface of blade upper surface.—Absent.

Leaf blade tip.—In plane of the leaf.

Undulation of margin.—Slight.

Apex.—Acuminate.

Thickness.—Medium.

Undulation of blade between main and lateral veins.—Slight.

Shape of teeth.—Conical, both sides convex.

Length of teeth.—Variable 3-10 mm.

Ratio length/width of teeth.—About 3:4.

General shape of petiole sinus.—Variable—open (ovate) to closed.

Tooth at petiole sinus.—Absent.

Petiole sinus limited by veins.—Absent.

Shape of upper lateral sinus.—Variable, ½ open, ½ ellipsoid.

Prostrate hairs between veins on lower surface of blade.—Absent.

Erect hairs between hairs on lower surface of blade.—Absent.

Prostrate hairs on main veins on lower surface of blade.—Absent.

Density of erect hairs on main veins on lower surface of blade.—Sparse; found only at junctions of main veins.

Prostrate hairs on main veins on upper surface of blade.—Absent.

Autumnal coloration.—Yellowish: 5Y 8/10.

Upper surface:

Color.—Dark green: 7.5 GY 4/4 to 4/6.

Surface texture.—Smooth.

Surface appearance.—Dull.

Goffering of blade.—Absent.

Lower surface:

Color.—Dark green: 5GY 6/6 to 6/8.

Anthocyanin coloration of main veins on lower leaf surface.—Absent.

Glossiness.—Low.

Pubescence.—Absent.

Surface texture.—Smooth.

Surface appearance.—Semi-glossy.

Petiole:

Length of petiole.—12.3 cm.

Length of petiole compared to middle vein.—Considerably shorter.

Density of prostrate hairs on petiole.—Absent.

Density of erect hairs on petiole.—Absent.

Shape of base of petiole sinus.—Half open, ovate.

Woody shoot:

Shape.—Stocky.

Internode length.—About 9.0 cm.

Width at node.—About 2.0 cm.

Cross section.—Circular.

Surface.—Smooth.

Main color Brown.—5YR 5/6.

Lenticels.—Inconspicuous.

Erect hairs on nodes.—Absent.

Erect hairs on internodes.—Absent.

Growth of axillary shoots.—Prolific.

Buds:

Shape.—Conical.

Cane bud fruiffulness.—Basal buds fruitful, 1-2 clusters per shoot.

### FLOWERS

General:

Flower sex.—Perfect.

Position of first flowering nodes.—3^{rd} or 4^{th} node.

Number of inflorescences per shoot.—1 or 2.

Date of full bloom.—May 9, 2001.

Time of bloom.—Early compared to other varieties growing in the area around McFarland, Calif.

### FRUIT

General:

Ripening period.—Early to mid-August at Delano, Calif.

Date of ripening.—About Aug. 10, 2001.

Use.—Fresh market.

Keeping quality.—Very good.

Resistance.—Insects: typical of Vitis vinifera. Diseases: typical of Vitis vinifera.

Shipping quality.—Good.

Date of first harvest.—Aug. 10, 2001.

Solids-sugar.—High, about 20 brix at full maturity.

Refractometer test.—24.0 brix.

Cluster:

Bunch size.—Large.

Bunch length (peduncle excluded).—About 27 cm.

Bunch width.—About 8 cm.

Bunch weight (natural).—1291 g.

Bunch weight (gibberellic acid treated).—1,272 g.

Bunch density.—Loose.

Number of berries.—75.

Form.—Elliptic.

Peduncle:

Length of peduncle.—7 cm.

Lignification of peduncle.—None.

Color.—5GY 5/10.

Berry:

Size.—Medium.

Uniformity of size.—Variable.

Berry weight (natural).—7.0 g.

Berry weight (gibberellic acid treated).—9.4 g.

Shape.—Elliptic to oblong.

Presence of seeds.—Rudimentary.

Cross section.—Circular.

Dimensions.—Longitudinal axis about 2.5 cm.; horizontal axis about 1.7 cm.

Skin color (without bloom).—Light green: 2.5GY 8/6 to 8/8.

Coloration of flesh.—Clear.

Juiciness of flesh.—Very juicy.

Berry firmness.—Very firm.

Particular flavor.—Neutral, typical vinifera.

Bloom (cuticular wax).—Strong.

Pedicel length.—12 mm.

Berry separation from pedicel.—Moderate.

Skin:

Thickness.—Thick.

Texture.—Tender.

Reticulation.—Absent.

Roughness.—Absent.

Tenacity.—Tenacious to flesh.

Tendency to crack.—Resistant.

What is claimed is:

1. A new and distinct variety of TABLE GRAPE cv. ‘13-21-12’ herein described and illustrated and identified by the characteristics enumerated above.
